(19) United States
(12) Patent Application Publication
(10) Pub. No.: US 2005/0096392 A1
(43) Pub. Date: May 5, 2005

(54) Use of creatine pyruvate for increasing stamina during highly intensive intermittent physical exertion

(76) Inventors: Ralf Jager, Freising (DE); Martin Purpura, Bonn (DE); Dirk Bokenkamp, Werne (DE)

Correspondence Address: FULBRIGHT & JAWORSKI, LLP, 666 FIFTH AVE, NEW YORK, NY, 10103-3198, US

(21) Appl. No.: 10/510,173

(22) PCT Filed: Apr. 3, 2003
(86) PCT No.: PCT/EP03/03501

(30) Foreign Application Priority Data
Apr. 5, 2002 (DE) 102 15 007.9

(51) Int. Cl. A61K 31/205

(52) U.S. Cl. 514/554

(57) ABSTRACT

The invention relates to the use of creatine pyruvate for increasing stamina during highly intensive intermittent physical exertion. Said use is to be applied in particular during short-term intensive muscular exertion and/or muscular exertion of short-duration and/or that is repeated in short intervals, preferably during sprinting and sporting performances in running disciplines and during exercises using sports equipment provided with rollers, wheels or sliding surfaces, in addition to during raising, pulling and/or lifting movements of the extremities and the neck. The invention relates in particular to variants, in which muscular exertion is maintained for between 0.1 seconds and 5 minutes and/or during which the muscular exertion is carried out with a frequency of between 0.1 and 600 minutes. In the latter variant, the muscular exertion can increase with each interval. The creatine pyruvate is administered in the required application in daily doses of between 500 mg and 30 g and in particular over a period of between 1 day and 12 weeks in powder, lozenge, capsule or coated tablet form, in liquids and also as a food supplement or functional food.

The present invention relates to a novel use of a creatine pyruvate to increase stamina.

Salts of pyruvic acid, pyruvates, have valuable physiological and therapeutic properties for treating various disorders, for example obesity and overweight, in the prevention of free radical formation, in particular but also to increase stamina.

According to the prior art, alkali metal and alkaline earth metal pyruvates are known, although sodium pyruvate and potassium pyruvate are unsuitable for therapeutic uses and as food supplement additives because of their content of sodium ions and potassium ions. Although magnesium pyruvates and calcium pyruvates are physiologically harmless, these salts have the corresponding disadvantage that they are not sufficiently storage stable, since magnesium and calcium ions greatly accelerate the decomposition of pyruvic acid and pyruvate ions. In this context, only calcium pyruvate monohydrate, as described in U.S. Pat. No. 5,962,734 and U.S. Pat. No. 6,342,631, exhibit significant advantages with respect to storage stability.

As already described, the use of pyruvates to increase stamina is sufficiently known. For instance, U.S. Pat. No. 6,221,836 describes the use of pyruvates in combination with an anabolic protein to increase the lean body mass or the muscle tissue. Furthermore, it is pointed out that pyruvates also increase stamina in athletic exercises. Pyruvyl-creatine adducts are also included with the pyruvates.

A composition consisting of calcium pyruvate and potassium pyruvate which is suitable for enteral administration is described in U.S. Pat. No. 6,008,252. This composition which is used to increase the muscle mass in mammals can additionally comprise pyruvyl-creatine adducts. This increase in muscle mass is achieved by daily exercises which are carried out under anaerobic conditions for a period of at least 20 minutes. Preference is given, however, to exercise periods of in each case more than 30 minutes, or more than 45 minutes. Examples of exercises under anaerobic conditions are training units on the weight bench, knee bends and pushups.

European patent EP 894 083 discloses creatine pyruvates of the formula (creatine)ₓ(pyruvate)_{y}(H₂O)ₙ, where x=1 to 100 and y=1 to 10 and n=0 to 10. In the creatine pyruvates containing water of crystallization, the pyruvate anion can also be present in the 2,2-dihydroxy form. These creatine pyruvates have good storage stability and contain the physiologically harmless creatine cation. Creatine, as muscle energy source, is not only an endogenous substance in the body and a valuable food supplement, but it also has valuable therapeutic properties. In a number of scientific studies it has been found that the intake of creatine in physical training can lead to an increase in muscle mass and muscle performance. This increase in muscle performance due to creatine is found, however, only in generally short-lasting physical exertion; beneficial creatine effects in the sense of a long-lasting increase in stamina are not described.

In particular in sports or movement sequences which proceed under highly intensive intermittent exertion of body or muscle sections, increases in muscle mass and stamina become beneficially noticeable. Of the previous fields of use of the known pyruvates, only improvements in stamina in exertion taking place over relatively long time periods, and also an increase in long-term stamina are reported. Hitherto, nothing is known of an increase in stamina due to pyruvates in short-term muscle activities or short-lasting muscle exertion. Such an increase in stamina in short-lasting exertion due to pyruvates was also simply not to be expected, since such exertion peaks are in part subject to other physiological mechanisms than long-term exertion.

It was therefore an object of the present invention to provide a novel method for increasing stamina in short-term exertion.

This object has been achieved by using creatine pyruvate for increasing stamina in intermittent physical exertion, in particular in highly intensive intermittent physical exertion.

Hereinafter, “creatine pyruvate” is taken to mean all compounds which contain the creatine cation and the pyruvate anion, or the 2,2-dihydroxypropionate anion in the molar ratio 1:1, or approximately in the molar ratio 1:1, but also mixtures of this salt with creatine or pyruvic acid. These mixtures can contain creatine or pyruvic acid and “creatine pyruvate”, for example in a molar ratio of up to 100:1, preferably up to 20:1.

Surprisingly, it has been found that, with administration of creatine pyruvate, a significant reduction in muscle fatigue under highly intensive intermittent exertion occurs, which clearly deviates from the previously known strength-increasing effect. Furthermore, it was observed that in the administration of creatine pyruvate, no adverse effects on kidney and liver function and also on fat metabolism parameters occurs. In addition, despite a demonstrated deposition of water into the muscle tissue, no change in body fat content was observed, as is reported, for example, in the prior art for other pyruvates. Also, it has been found that the overacidification of muscle tissue, otherwise known from highly intensive intermittent exertion, does not occur, or only occurs to a very slight extent. Overall, these advantages were not to be expected.

The present invention thus relates to the use of creatine pyruvate in association with intermittent physical exertion. In this respect, the invention takes into account, in particular, intensive muscle exertion which is short-term and/or is short duration and/or those which are repeated in short time intervals. Particular preference is given to sprinting and sporting performances in the running area, exercises which are carried out on sporting equipment provided with rollers, wheels and/or sliding surfaces, and also raising, pulling and/or lifting movements of the extremities and neck. Especially, build-up and demonstration measures of the body's muscular system come into consideration, as occur, especially, in bodybuilding and in weightlifting. Furthermore, ball sports, such as basketball, volleyball, football, American football, baseball, hockey and handball come into consideration. The highly intensive intermittent physical exertion, however, can also occur in impact sports, such as (table) tennis, badminton, squash, ice hockey and lacrosse, in rowing (inter alia including kayak and canoe sports), in combat sports, such as wrestling, karate, judo, Tae-Bo, kickboxing and boxing, in cycling, in sledding sports, such as tobogganing, skeleton and bobsleigh sports, in fencing, swimming and skiing sports (here in particular mogul skiing and freestyle) and also in archery, in aerobic exercise and all forms of exercises related to and derived from these and also in shooting up movements.

The claimed use thus focuses on all activities which rapidly produce an intensive to maximum exertion of defined muscles or muscle groups.

The inventive use is advantageous in particular in muscle exertion which lasts in each case 0.1 to 5 minutes.

The use of creatine pyruvate likewise has a particularly beneficial effect in the context of the present invention if the muscle exertion takes place at a frequency of 0.1 to 600 per minute and preferably at a frequency of 3 to 120 per minute. The upper frequency limit can approach the typical tremor behavior of muscles.

As a further variant, the present invention covers muscular exertion which repeats after intervals of 1 second to 5 minutes. The intervals here can be of identical length or each of different length, with intervals of identical length being particularly preferred.

A preferred variant is also the use of creatine pyruvate in repeating muscle exertion the duration of which is of identical length.

The present invention thus covers a broad spectrum of highly intensive intermittent physical exertion as occurring in particular in movement sports, but especially in (top level) competitive sport.

In this context, the inventive use of creatine pyruvate can be considered to be particularly advantageous if it is performed with muscle exertion which increases from exertion period to exertion period, with the exertion being able to be increased to a maximum. Achieving the performance limit of the muscle tissue is, however, only to be considered as an exception in most cases. Customarily, in highly intensive intermittent exertion, at most 80 to 90% of the absolute performance maximum is achieved.

To cover all phenomena of the described intermittent physical exertion, the present invention comprises the use of creatine pyruvate in daily doses which are between 500 mg and 30.0 g. In particular, daily doses are to be recommended which are between 800 mg and 15.0 g, and in particular between 1.5 and 5.0 g.

Creatine pyruvate is preferably administered according to the invention over a period of at least one day and up to 12 weeks, although generally, depending on the training state of the muscle sets used in the exertion in each case, and including the “loading phase” of usually one week, 4 to 6 weeks being sufficient. Of course, the consumption period can also extend beyond the recommended 12 weeks and can be as long as desired without adverse health effects. The described effects in the context of a significant increase in stamina are due to the intake of creatine pyruvate, also, at any rate without the loading phase known from other compounds, that is to say they are also possible without “flooding”, which moreover, in contrast to the prominent creatine monohydrate, is successful with creatine pyruvate in low dose.

Finally, the creatine pyruvate in the context of the present invention can also be administered together with other physiologically active, and in particular exogenous, compounds, compounds such as caffeine, creatine monohydrate or creatine salts and derivatives differing from creatine pyruvate, protein, amino acids such as arginine, L-glutamine and carnitine and derivatives thereof, fats, such as linolenic acid and conjugated linoleic acid, and phospholipids, such as phosphatidylcholine and phosphatidylserine, carbohydrates such as diacylglycerol, glycerol and ribose, vitamins, minerals and sweeteners, pyruvate derivatives differing from creatine pyruvate (inorganic and organic pyruvates and derivatives thereof), ketoacids, such as β-hydroxy-β-methylbutyrate (HMB), buffer compounds, for example sodium hydrogencarbonate and any mixtures thereof, being particularly preferred.

The creatine pyruvate can be used in powder, tablet, capsule or dragée form, but also in liquids, as a food additive and/or food supplement and/or as a functional food, or in other administration forms.

With the inventive use of creatine pyruvate, a novel possibility for increasing stamina in highly intensive intermittent physical exercise, which occurs especially without negative consequences on important physical and metabolic functions, reduces or completely prevents overacidification phenomena, for example aching muscles, and leads to no adverse changes in the fat mass.

The examples hereinafter explain the advantages of this novel use of creatine pyruvate.

### EXAMPLES

In a double-blind study, creatine pyruvate (A) and placebo (B) were tested. The male experimental subjects (n=32; age: 18 to 32) were divided into groups, each of n=16, so that the maximum oxygen consumption per kilogram of body weight was on average the same in the groups, and the types of sport undertaken were usually distributed the same as far as possible. This led to the fact that the mean strength in the intermittent studies was not the same. The test subjects of group A were administered 5.0 g of creatine pyruvate per day over a period of 28 days. The initial test (IT) was carried out on the 1st day, and the closing test (CT) on the 29th day.

Course of the Typical Experimental Day:

In the morning between 8 and 11 o'clock fasted blood sampling, record of anthropometric data; standardized breakfast; then intermittent test of the lower arm musculature.

Anthropometric Data (Tab. 1):

The body weight increased significantly in the members of group A. The body fat content (by 2 different methods: skinfold thickness, and also BIA) remained the same in groups A and B. Since the weight increased and the fat mass remained the same, an increase in the amount of water in the body could be concluded. For a one-sided test, the increases in body H₂O were significant (A: p<0.05). The circumference of the thickest part of the lower arm increased in group A (p<0.005). The same applied to the circumference at the epicondyles (p<0.005).

Fasted Values (Tab. 2):

The number of erythrocytes decreased significantly in group A (p<0.02). In contrast, HB and Hct were not different. There was no difference in group B. There was no difference in leukocyte number in any group.

There were further significant changes in group A only in creatinine (p<0.001) and urea contents (p<0.01).

The increase of creatinine and simultaneous decrease of urea concentration to a significant extent in each case are assessed as an index of a decreased purine conversion. This is also shown in a reduced uric acid concentration. Under these conditions, a t-test for a one-sided test is permitted: this gave a significant decrease (p<0.05) for the uric acid concentration in group A.

The choline esterase increased significantly in placebo group B (p<0.05). The reduction of LDL cholesterol in group A was just above the level of significance (p>0.07).

Evaluation:

Hb and Hct were unchanged in groups A and B. The administration of creatine pyruvate thus had no effect on fat metabolism at rest. Creatine pyruvate may have been able to reduce ATP breakdown during the day, which can lead to intramuscular ATP concentrations.

Intermittent Test with the Lower Arm Musculature (Inventive Example):

Method:

The arm of the seated test subjects is positioned extended (horizontally) at the side at shoulder height. The hand lies on an adjustable-weight gripper (stroke length: 3 cm). The arm is supported at the elbow. The test subjects must perform highly intensive intermittent work, for which they compress the adjustable-weight gripper at the maximum possible frequency. The weight in the basket is 80% of the maximum weight achieved in a preliminary test in which the load, starting at 7.5 kg, is increased by 2.5 kg every 3 minutes. The contraction frequency is 24/min. The maximum weight is reached when the 3 cm stroke length can no longer be overcome. Blood is taken from the cubital vein on the working arm. The skin blood flow is reduced by cooling. Blood was taken in each case before and after the 1st, 2nd, 6th, 9th and 10th period.

Evaluation of the Mechanical Parameters:

Via an inductive distance measurer on the adjustable-weight gripper, the stroke length of the weight basket was measured. From the signal, 4 measurements can be obtained.
1. Contraction rate
2. Stroke length
3. Duration of the total contraction
4. Integral of stroke length over time

From these data and the weight of the basket, the force, power and work can be calculated. The force is the mean force during the shortening phase, not the point-measurement maximum force. It is only the force which was developed additionally to the force necessary to overcome the force of gravity. In addition, the relaxation rate and the contraction frequency were determined. The evaluation was started with the fourth contraction. Thereafter, each contraction was evaluated over 12 seconds. The mechanical parameters were determined in the 1st, 2nd, 6th and 9th interval.

Creatine pyruvate led to the following significant changes (analysis of variance):

The contraction frequency (p<0.01), force (p<0.01), power calculated from the shortening rate (p<0.005) was increased in all intervals. The relaxation rate showed a tendency to increase.

Although slight improvements were found in the placebo group, they were not significant.

Assessment:

Creatine pyruvate produces a significant increase in performance which is found not only in short exertion, but which is still present even in the final intervals. Creatine pyruvate therefore reduces fatigue and increases endurance in highly intensive exertion. The increase in frequency can in part be correlated with the increase in relaxation rate.

Blood Tests:

For the parameters which are not associated with the acid-base status, there was no significant difference between initial test and closing test of groups A and B.

If the maximum values during exertion are compared (t test for paired random samples), after administration of creatine pyruvate, there is a significantly lower rise (p<0.002) during exertion. In B, no significant differences were observed.

Acid-Base Status:

In groups A and B, there were no significant differences between the initial and closing tests in the absolute values of pH, pCO₂, lactate and BE. The differences from the blood sample before exertion also did not differ significantly among the different preparation administrations. The change of pCO₂ during the working phase in group A had a tendency to be greater. When all pCO₂ differences between measurements before and after intervals 2, 6, 9 and 10 were pooled, there was a significantly (p<0.05) greater difference in group A.

The HBO₂ was significantly lower at the end of the interval pauses in group A.

Assessment:

The reduced NH₃ concentration indicates a stabilization of the ATP concentration under exertion after creatine pyruvate administration, and the tendency towards lower HBO₂ indicates an increased oxidative metabolism. The low HBO₂ values at the end of the pauses can be assessed firstly as a consequence of the greater energy conversion during the interval; secondly they can also indicate a more rapid recovery in the pause and therefore greater fitness. The greater changes in pCO₂ during the 15-second work can be due to an increased oxidative metabolism and they can be due to an increased intracellular buffering against protons by creatine phosphate breakdown.

Summary of the Results

Creatine pyruvate clearly has a performance-enhancing effect. In the case of highly intensive exertion, it additionally enhances stamina. The change in relaxation rate after administration of creatine pyruvate is surprising. Since only a few of the measured parameters in blood changed significantly, it must be assumed that a plurality of small changes complement one another and thus cause the performance enhancement. Decreased ATP breakdown during the intensive exertion, an enhancement of intracellular buffering against protons and an enhancement of oxidative metabolism must be seen as participating factors.

TABLE 1

| | | IT¹⁾ | — | CT²⁾ | — | | Significance |
|---|---|---|---|---|---|---|---|
| Group | Measurement parameter | mean | SD | mean | SD | n | t test |
| A | Body weight [kg] | 81.70 | 10.90 | 83.20 | 10.70 | 16 | p < 0.001 |
| | BIA fat measurement [kg] | 16.69 | 4.45 | 16.63 | 4.64 | 16 | n.s. |
| | BIA water measurement [kg] | 46.64 | 3.99 | 47.39 | 3.62 | 16 | n.s. |
| | Body fat (skinfold) [%] | 11.33 | 2.17 | 11.59 | 1.37 | 8 | n.s. |
| | Circumference of lower elbow [cm] | 28.94 | 2.09 | 29.62 | 2.21 | 14 | p < 0.004 |
| | Circumference of thickest point of the arm | 29.01 | 2.17 | 29.67 | 2.21 | 14 | p < 0.006 |
| | [cm] | | | | | | |
| B | Body weight [kg] | 77.60 | 7.28 | 77.70 | 7.31 | 17 | |
| | BIA fat measurement [kg] | 15.54 | 5.37 | 14.81 | 4.38 | 14 | n.s. |
| | BIA water measurement [kg] | 45.14 | 2.00 | 45.61 | 3.53 | 14 | n.s. |
| | Body fat (skinfold) [%] | 10.09 | 2.36 | 10.11 | 2.14 | 10 | n.s. |
| | Circumference of lower elbow [cm] | 28.27 | 1.20 | 28.49 | 1.12 | 15 | n.s. |
| | Circumference of thickest point of the arm | 28.32 | 1.21 | 28.58 | 1.15 | 15 | n.s. |
| | [cm] | | | | | | |

¹⁾Initial test
²⁾Closing test

TABLE 2

| | | IT¹⁾ | — | CT²⁾ | — | | Significance |
|---|---|---|---|---|---|---|---|
| Group | Measurement parameter | mean | SD | mean | SD | n | t test |
| A | PT | 5.40 | 0.95 | 6.20 | 1.17 | 15 | p < 0.013 |
| | Urea | 34.07 | 4.36 | 30.00 | 4.97 | 15 | p < 0.008 |
| | Uric acid | 5.64 | 0.88 | 5.29 | 0.77 | 15 | n.s. |
| | Creatinine | 1.07 | 0.06 | 1.19 | 0.08 | 15 | p < 0.000 |
| | Leukocytes | 8980.00 | 2454.7 | 9386.67 | 2363.58 | 15 | n.s. |
| | Erythrocytes | 5.30 | 0.40 | 4.96 | 0.46 | 15 | p < 0.010 |
| | Hb | 15.56 | 0.73 | 15.40 | 0.72 | 16 | |
| | Hct | 45.50 | 2.14 | 45.13 | 2.71 | 16 | |
| B | PT | 7.06 | 3.73 | 9.06 | 6.65 | 17 | n.s. |
| | Urea | 32.76 | 9.17 | 31.71 | 10.16 | 17 | n.s. |
| | Uric acid | 4.98 | 1.09 | 5.03 | 0.96 | 17 | n.s. |
| | Creatinine | 1.05 | 0.10 | 1.06 | 0.10 | 17 | n.s. |
| | Leukocytes | 9435.71 | 1810.92 | 10107.14 | 2166.12 | 14 | n.s. |
| | Erythrocytes | 5.08 | 0.40 | 5.14 | 0.43 | 14 | n.s. |
| | Hb | 14.74 | 0.92 | 14.76 | 0.83 | 17 | |
| | Hct | 43.27 | 3.46 | 42.42 | 1.94 | 17 | |

¹⁾Initial test
²⁾Closing test

1. A method of increasing stamina during intermittent physical exertion comprising administering creatine pyruvate to a subject in need thereof.

2. The method of claim 1 wherein during short-term intensive muscular exertion and/or muscular exertion of short duration and/or that is repeated in short intervals, preferably during sprinting and sporting performances in running disciplines and during exercises on sporting equipment equipped with rollers, wheels or sliding surfaces, and also during raising, pulling and/or lifting movements of the extremities and neck, very particularly preferably during build-up and demonstration measures of the body's muscular apparatus, in ball sports, in impact sports, in rowing sports, in combat sports, in cycling, in sledding sports in fencing, swimming and skiing sports, in archery, in aerobics and in shooting up movements.

3. The method as claimed in claim 1, wherein the exertion lasts for 0.1 second to 5 minutes.

4. The method as claimed in claim 1, wherein the muscular exertion occurs at a frequency of 0.1 to 600 per minute.

5. The method as claimed in claim 1, wherein the muscular exertion repeats after intervals of 1 second to 5 minutes.

6. The method as claimed in claim 1, wherein the duration of the repeating muscular exertion is of equal length.

7. The method as claimed in claim 1, wherein the muscular exertion increases from exertion interval to exertion interval, particularly preferably to the maximum.

8. The method as claimed in claim 1, wherein creatine pyruvate is administered in daily doses of 500 mg to 30.0 g.

9. The method as claimed in claim 1, wherein creatine pyruvate is administered over a period of 1 day to 12 weeks.

10. The method as claimed in claim 1, wherein creatine pyruvate is used together with another physiologically active compounds.

11. The method as claimed in claim 1, wherein said creatine pyruvate is administered in powder form, tablet form, capsule form or dragée form, in liquids, as a food additive and/or food supplement and/or functional food.

12. The method of claim 4, wherein the muscular exertion occurs at a frequency of 3 to 120 per minute.

13. The method of claim 1, wherein the muscular exertion repeats after identical intervals.

14. The method of claim 5, wherein the muscular exertion repeats after identical intervals.

15. The method of claim 9, wherein the creatine pyruvate is administered daily.

16. The method of claim 10, wherein said physiologically active compound is an exogenic compound.

17. The method of claim 10, wherein said another physiologically active compound is selected from the group consisting of caffeine, creatine monohydrate or creatine derivatives different from creatine pyruvate, protein, amino acids and derivatives thereof, fats and phospholipids, carbohydrates, vitamins, minerals and sweeteners, pyruvate derivatives different from creatine pyruvate, keto acids, buffer compounds and mixtures thereof.
